(19) United States
(12) Patent Application Publication
(10) Pub. No.: US 2018/0055849 A1
(43) Pub. Date: Mar. 1, 2018

(54) NUTRACEUTICAL CAPSULE AND TABLET FORMULATIONS PROVIDING ENHANCED MENTAL CLARITY, CONCENTRATION AND STAMINA WHILE MINIMIZING ADRENALINE AND DOPAMINE CONCENTRATION PERTURBATIONS ASSOCIATED WITH WITHDRAWAL

(71) Applicant: Power Supplements, LLC., Houston, TX (US)

(72) Inventors: Jason Kelly Allison, Houston, TX (US); Guillermo A. Amtmann, Houston, TX (US); John Burton Steele, Houston, TX (US)

(21) Appl. No.: 15/621,622

(22) Filed: Jun. 13, 2017

Related U.S. Application Data
(63) Continuation of application No. 15/369,330, filed on Dec. 5, 2016, now abandoned.
Application No. 15/369,330 is a continuation-in-part of application No. 15/225,391, filed on Aug. 1, 2016, now abandoned.

(51) Int. Cl. A61K 31/522; A61K 9/20; A61K 31/137; A61K 31/455; A61K 31/706; A61K 31/197; A61K 31/675; A61K 31/4188; A61K 31/519; A61K 31/714; A61K 36/74; A61K 9/00; A23L 33/105; A23L 33/15; A23L 2/52; A23L 33/10

(52) U.S. Cl. CPC A61K 31/522; A23V 2002/00; A61K 31/137; A61K 31/455; A61K 31/706; A61K 31/197; A61K 31/675; A61K 31/4188; A61K 31/519; A61K 31/714; A61K 36/74; A61K 9/0053; A23L 33/105; A23L 33/15; A23L 2/52; A23L 33/10; A61K 9/2095

(57) ABSTRACT

The invention describes one or more compositions that are provided in capsule or tablet form comprising at least caffeine, hordenine and β-phenylethylamine for increased mental clarity and stamina. The caffeine is often provided from green coffee bean extracts and normally comprises from 0.0001 g to 0.30 g of caffeine from 0.00001 g to 0.1 g of hordenine from barley and from 0.00001 g to 1.0 g β-phenylethylamine (or synthetic source) from cocoa beans. The present invention provides a synergistic effect delivering extended mental acuity, mental stamina, energy and motivation.

### PRIORITY

This application is a Continuation of and claims priority under 35 USC 120 of U.S. application Ser. No. 15/369,330 filed Dec. 5, 2016, entitled “NUTRACEUTICAL CAPSULE AND TABLET FORMULATIONS PROVIDING ENHANCED MENTAL CLARITY, CONCENTRATION AND STAMINA WHILE MINIMIZING ADRENALINE AND DOPAMINE CONCENTRATION PERTURBATIONS ASSOCIATED WITH WITHDRAWAL”, which is a Continuation-In-Part and claims priority under 35 USC 120 of U.S. application Ser. Number 15/225,391 filed Aug. 1, 2016, entitled “Synergistic Nutraceutical Beverage Formulations Providing Enhanced Thermogenesis, Mental Clarity and Stamina While Minimizing Adrenaline and Dopamine Concentration Perturbations Associated With Withdrawal”. The entire contents of the application are hereby incorporated by reference.

### FIELD OF INVENTION

The present disclosure relates to nutritional and nutraceutical products, particularly to formulations in capsule form providing nutritional components that improve both physical and mental aspects of the human condition. More specifically, the capsule components in this disclosure are designed to boost focus as well as increase mental concentration and stamina while also (and in some cases) simultaneously reducing or eliminating perturbations existing during adrenaline and dopamine withdrawal. Perturbations are defined as rapid changes in concentration levels over relatively short time periods. In this case short time periods for human consumption are normally less than 5-6 hours.

### BACKGROUND

A nutraceutical is a food, food product or dietary supplement that provides health and medical benefits, including the prevention, treatment, and enhancement of the human condition and in many cases, for the benefit of mammals in general. A “dietary supplement” is a product that contains nutrients from food products that are concentrated in capsule or tablet form. Most vitamins are made from “food grade” chemicals but a nutraceutical is generally made from pharmaceutical grade chemicals of higher purity and with more predictable and consistent results. Thermogenesis is a term which literally means “the creation of heat”. Often, the term thermogenesis is used as a synonym for the term “energy”. Energy is transformed, converted, transported, and stored within the human body. Human energy comes into the body from what is normally consumed in the form of food and/or beverage. In many cases, beverages contain food-based substituents including vitamins, minerals, and additional supplements which are contained in many of the foods we consume. One unit of the measurement of heat energy involves the use of the terms “joules or kilocalories” and often kilocalories are counted on food and beverage labels in terms of “calories”. Specifically, a calorie is the amount of heat needed to heat one gram of water one degree Celsius, Calories are the unit of measurement describing how much energy is stored in a food or beverage. Water does not contain any calories, however it is normally found in the food and beverages consumed by humans, and as we consume these substances, energy is either transformed or stored.

As energy is transported through the body's system, some energy leaves the body as fecal energy. Other energy is lost through the urinary tract. Energy not lost through waste by these two means is available for ensuring at least baseline (or basal) metabolic functions. One of the largest energy expenditures in the human body is thermic (heat) energy. Thermic energy differentiates an endoderm (mammal) from an ectoderm (reptile). The endoderm's basal metabolism is 8 to 10 times higher than that of most ectoderms. Therefore, mammals, and in particular humans, utilize tremendous amounts of the energy that is converted into thermic energy. Energy not used for thermic energy is then available as net energy for the body's cellular reproduction, growth (especially in children), work (muscle movement), and storage. The most common storage form of thermic energy is known as fat.

There are at least three forms of thermogenesis. The first form is work-induced from exercise. It is necessary for human muscles to create or utilize heat in order to work more effectively than when the muscles are cold.

A second form of thermogenesis is known as thereto-regulatory thermogenesis. This form provides for keeping the temperature of the human body regulated. The average body temperature is 98.7 degrees (F.). In addition, there are two types of thermo-regulatory thermogenesis: shivering and non-shivering. Shivering helps the body to create heat. The skeletal muscles create the shivering. There's a small amount of muscle on each hair that can regulate heat gain and loss.

Non-shivering thermogenesis fits the third classification form, which is also known as diet-induced thermogenesis. Eating a meal or drinking other than water, will produce diet-induced thermogenesis. Normally, humans require more energy to digest food and/or beverages than are being consumed. There are regions within the body that can measure the most critical phenomena regarding the purpose of this disclosure; namely non-shivering or diet-induced thermogenesis.

Diet-induced thermogenesis is very important in animals that hibernate, such as bears, or small animals with a very large surface area in comparison with their body weight. Brown adipose or “brown fat” tissue is where a significant amount of the energy is stored in the body. “Brown fat” is also very prevalent in newborn babies as they exhibit tremendous amounts of non-shivering thermogenesis to regulate their body temperature. As humans age, this system depletes somewhat but remains an important part of survival.

Brown fat (adipose tissue) is located around blood vessels and major organs. When it is actively triggered, it causes the warming of the blood. Warm blood is circulated throughout the body to spread this energy in the form of heat for warming the entire body. The body's thermogenic system triggers the sympathetic nervous system. Under conditions of cold or when larger portions of calories are consumed, the hypothalamus gland registers this energy consumption and then triggers the sympathetic nervous system (also known as an automatic nervous system). The sympathetic nervous system controls many vital functions including heartrate, blood pressure (the circulatory system) and breathing (the respiratory system). Thus, the autonomic (involuntary) nervous system continuously requires and consumes energy.

In the area of the body where the nerves transmit biothemical signals via pathways, one of these neuro “transmitters” is known as norepinephrine. The triggering of the sympathetic nervous system causes the release of norepinephrine from a nerve terminal across the synapse which subsequently binds to receptors to propagate a nerve impulse.

This process increases (in some cases rapidly and dramatically) thermogenesis as well as other activity related functions generated by the nervous system. One way to visualize these somatic (body) functions is by likening it to raising the body's thermostat. All humans have a basal metabolic rate which can be measured and indicates (in a rather precise manner), the energy required for maintaining vital functions. It is possible, by using formulations described in the present disclosure, to modulate or raise the metabolic rate above the basal rate.

Sweating or perspiration is often associated with one symptom of this increase in thermogenesis. Sweating occurs when more energy is used during thermogenesis of the sympathetic system than the energy used for work related activities. In the case of sweating, less energy is stored and less energy is converted to fat as opposed to when the body is operating at its basal metabolic rate. In this case, less brown fat is activated, thereby further reducing the metabolism of white fat cells, which are the primary fat storage depository in the human body. It is worth noting that although humans have a consistent number of fat cells, the size can greatly vary. The number of fat cells remains the same, but the size changes.

The brown fat cell is unique in its mitochondrion. Mitochondria are the “energy factories”—oxidative phosphorylation sites—for adenosine triphosphate production (ATP). In the brown fat cell, unique mitochondria help create energy via ATP synthesis. The brown fat cell is also an energy consumer, and it is heat energy that is being consumed. It has been postulated that much of human obesity is less associated with eating habits than it is with brown fat cell deterioration. If these cells deteriorate, energy use and release can be severely compromised. Brown fat activity studies are underway which indicate that post-obese people have a deficiency in their brown fat system.

When an intentionally induced form of thermogenesis is created, by for example, the use of nutritional or nutraceutical supplementation with food or beverages, people feel they have more energy. Often they are generally able to perform more work related operations (both physical and mental), which also leads to an increased utilization of fat as the stored calories are converted into released calories. When calories released from the body exceed those consumed, body weight is reduced.

In the past, many of the types of formulations that were designed to provide increased and improved mental stimulation and stamina have also provided deleterious side effects or “symptoms”. The capsules of this disclosure have been carefully and precisely formulated to reduce and in some cases completely eliminate these deleterious, unwanted side effects or symptoms.

### SUMMARY

Generally, the present disclosure describes synergistic nutraceutical capsule formulations providing enhanced/improved mental concentration and stamina while minimizing adrenaline and dopamine withdrawal. In most cases these synergistic effects are simultaneous within the consumer's body and are primarily due to the ability to slow the metabolic rate of caffeine and β-phenylethylamine withdrawal when combined with the MAO-B inhibitor, hordenine HCL. It is also clear that combining caffeine, β-PEA, and hordenine (likely) results in a synergistic residual reduction in the withdrawal symptoms associated with catecholamine withdrawal following consumption of the capsule formulation(s) due to the increased half-life of β-PEA. These symptoms are more fully described below.

In order to extend physical and mental stamina and (in some cases) simultaneously reduce unwanted withdrawal symptoms, the present disclosure describes a capsule combination of caffeine, hordenine, and β-phenylethylamine (β-PEA) in specific doses. This capsule stimulates and enhances brain function related to mood and focus, while providing an increase in thermogenesis associated with a feeling of increased energy due to increased physical metabolic activity.

A capsule or tablet of the formulation is normally more highly concentrated with respect to caffeine, hordenine, β-PEA and includes excipients, or fillers, necessary for tablet production. Liquid formulations normally provide these “active” ingredients with no excipients. Here the term “active” or “actives” is meant to convey the meaning of ingredients that are known to provide active stimulation of thermogenesis and/or brain activity when ingested by humans. Alternatively, the term “inactive”, “inactives” “excipients” or “filler(s)” refer to, for example, a simple carrier or binding agent, primarily inert fillers such as calcium, magnesium, or sodium salts, thickening agents, emulsifying agents, and buffering agents.

The concentrations, amounts, and other numerical ranges associated with the use of these inactives/excipients/fillers are merely for convenience and brevity and should be interpreted flexibly to include not only the numerical values explicitly recited as the limits of the range, but also to include all the individual numerical values or sub-ranges encompassed within that range as if each numerical value and sub-range is explicitly recited.

Depending on the composition or formulation, other excipients may be used as would be apparent to those skilled in the art. For example, example compositions may include tablets that can be coated on the outside for ease of swallowing by a human or other mammals. If tablets or other formulations are produced without a coating, it may be desirable to add one or more flavoring agents for example, as would be apparent to those skilled in the art. By way of further example, liquid compositions for example, may require one or more carriers.

For the purposes of this disclosure an excipient is defined as a substance formulated alongside the active ingredient of a supplement, included for the purpose of long-term stabilization, bulking up solid formulations that contain potent active ingredients (thus often referred to as “bulking agents”, “fillers”, or “diluents”), or to confer a therapeutic enhancement on the active ingredient in the final dosage form, such as facilitating absorption, reducing viscosity, or enhancing solubility.

Excipients can also be useful in the manufacturing process, to aid in the handling of the active substance concerned such as by facilitating powder flowability or non-stick properties, in addition to aiding in vitro stability such as prevention of denaturation or aggregation over the expected shelf life. The selection of appropriate excipients also depends upon the route of administration and the dosage form, as well as the active ingredient and other factors.

Types of excipients include antiadherents, binders, fillers, diluents, coatings, colors, flavors, disintegrants, glidants, lubricants, plastifying components, preservatives, sorbents, sweeteners, vehicles (carriers) and other excipients known to those skilled in the art, depending e.g., on the composition being formed, method of formation, active ingedient(s) being used, etc.

Antiadherents reduce the adhesion between the powder and the punch face used during tablet manufacture and to protect tablet from sticking. The most commonly used antiadherent is magnesium stearate.

As used herein, the term “binder” is intended to encompass binders known to those skilled in the art. The following is a list of non-limiting example embodiments of natural binders, as provided in U.S. Pat. No. 8,147,882, that may be used in accordance with various embodiments herein: acacia, alginic acid, carbomer (e.g., carbopol), carboxymethylcellulose sodium (CMC), dextrin, ethyl cellulose, gelatin, guar gum, hydroxyethyl cellulose (HEC), hydroxypropyl cellulose (e.g., Klucel) (HPC), hydroxypropyl methyl cellulose (e.g., Methoce HPMC), magnesium aluminum silicate, maltodextrin, methylcellulose, magnesium stearate, pregelatinized starch, sodium alginate, starch and zein. As with other excipients herein, the amount of the binder may vary depending on various factors as would be known or can be determined b those skilled in the art.

As used herein, the terms “filler” and “diluent” are intended to encompass fillers known to those skilled in the art. The following is a list of non-limiting example natural fillers, as provided in U.S. Pat. No. 8,147,882, that may be used in accordance with various embodiments herein: microcrystalline cellulose, dextrose, calcium phosphate anhydrous, calcium carbonate, calcium sulfate, compressible sugars, dextrates, dextrin, dibasic calcium phosphate dihydrate, glyceryl palmitostearate, hydrogenated vegetable oil (type I), kaolin, lactose, magnesium carbonate, magnesium oxide, maltodextrin, mannitol, potassium chloride, powdered cellulose, pregelatinized starch, sodium chloride, sorbitol, starch, sucrose, sugar spheres, talc and tribasic calcium phosphate. The amount of the filler or diluent may vary depending on various factors as would be known or can be determined by those skilled in the art.

According to US Patent Application Number 2013/0296445, the term “natural lubricant” is a material having a lubricant effect, which is prepared by a physical process without carrying out a chemical extraction process or a chemical reaction. Pharmaceutically acceptable lubricants, for example, including, but not limited to, magnesium stearate, polyethylene glycol, talc, calcium stearate, microcrystalline cellulose, etc., are defined as chemical synthetic products, or synthetic lubricants, for which bean powder having a crude fat content of 10-25% is substituted for the synthetic additive. U.S. Pat. No. 8,147,882 states the following is a list of non-limiting example embodiments of lubricants that may be used in accordance with various embodiments herein: magnesium stearate, stearic acid, calcium stearate, glyceryl monostearate, glyceryl palmitostearate, hydrogenated castor oil, hydrogenated vegetable oil, light mineral oil, mineral oil, polyethylene glycol, sodium benzoate, sodium lauryl sulfate, sodium stearyl fumarate, talc and zinc stearate. The amount of the lubricant may vary depending on various factors as would be known or can be determined by those skilled in the art.

US Patent Application Number 2007/0059358, provides pharmaceutically acceptable matrix adjuvants selected from a group consisting of monosaccharide, oligosaccharide, polysaccharide, sugar ester, sugar alcohol, alpha-hydroxy acid, higher fatty acid derivative, higher aliphatic alcohol, polyol, urea, and poly(ethylene oxide) derivative; and further providing that the monosaccharide is D-ribose, fructose, glucose, xylose; the oligosaccharide is trehalose, raffinose, maltose; the polysaccharide is gelose; the sugar ester is sucrose ester, D-ribonic acid-γ-lactone; the sugar alcohol is erythritol, sorbitol, xylitol, arabitol, isomaltitol, lactitol; the alpha-hydroxy acid is malic acid, citric acid; the higher fatty acid derivative is sodium stearate, glycerin stearate, glycerin palmitate, shellac; the higher aliphatic alcohol is cetyl alcohol, stearyl alcohol; the polyol is phenyl ethanediol; the poly(ethylene oxide) derivative is polyoxyethylene monosteatate, and polyoxyethylene alkyl ether. The matrix adjuvants include the adjuvants mainly derived from natural materials, especially from plants. The matrix adjuvants may optionally include a plastifying component.

According to US Patent Application Number 2007/0059358, natural plastifying components can be selected from the group consisting of starch and their derivatives, cellulose and their derivatives, arabic gum, dextran, chitin, sesbania gum, carrageen gum, Indian gum, danish agar, tragacanth gum, carrageenin, tamarind gum, pectin, xanthan gum, alginic acid and the salts thereof, dextrin, cyclodextrin, agar, lactose, carbomer, poloxamer, silicon dioxide, glutin, glycerin monostearate, and polyoxyethylene monostearate. More preferably, the plastifying component may be selected from the group consisting of pregelatinized starch, carboxymethyl starch, methyl cellulose, sodium carboxymethyl cellulose, hydroxypropyl methyl cellulose, arabic gum, alginic acid, dextrin, cyclodextrin, agar, lactose, glycerin monostearate, polyoxyethylene monostearate, cross-linked sodium carboxylmethyl cellulose, and silicon dioxide.

All references provided herein are hereby incorporated by reference in their entirety. In one embodiment, these capsule formulations utilize only components and compounds extracted or derived from earth grown or naturally occurring earth-available substances.

In another embodiment, the composition will optionally contain calcium and magnesium salts. Calcium and magnesium salts can be present in amounts ranging from about 0.001 g to 0.50 g,

More specifically, the present disclosure describes one or more nutraceutical capsule formulations providing enhanced thermogenesis, mental concentration, and stamina to mammals, comprising at least three distinct synergistic components including caffeine, β-phenylethylamine, and hordenine, wherein the formulations reduce or eliminate undesirable side effects during or after mammalian ingestion of the capsule formulation(s).

For these capsule formulations mammals are humans wherein hordenine is hordenine HCl.

In another embodiment the capsule formulations comprise β-phenylethylamine that is β-phenylethylamine HCl.

In another embodiment, the synergistic components act to increase catecholamine and indoleamine concentrations and the synergism is accomplished as the catecholamine and indoleamine concentrations cross a blood-brain barrier after consumption, thereby reducing or eliminating acute symptoms associated with rapid decline of the catecholamine and indoleamine concentrations in the blood.

In another embodiment, these synergistic components undergo a metabolic breakdown resulting in a controlled decrease in the catecholamine and indoleamine concentrations such that the concentrations revert to a pre-existing basal level associated with the consumer of the capsule formulation.

These synergistic components provide synergy in that β-phenylethylamine decreases serotonin reuptake, hordenine prevents metabolic breakdown of β-phenylethylamine and maintains serotonin levels, while caffeine decreases serotonin levels.

The synergistic component, β-phenylethylamine corrects for reduction of serotonin levels that occur following prolonged consumption of caffeine.

In another embodiment, the caffeine used for these beverages is a green coffee bean extract.

In a separate embodiment, one or more nutraceutical capsule and/or tablet formulations providing enhanced thermogenesis, mental concentration, and stamina to mammals, comprising at least three distinct synergistic components including caffeine, β-phenylethylamine, and hordenine, provide a boost of energy-generating catecholamines from the caffeine including dopamine and norepinephrine which produces amphetamine stimulation and performance enhancement. In this case, the β-PEA further promotes an efflux of catecholamines which blocks a re-uptake and simultaneously decreases reuptake of serotonin. This decrease may or may not be simultaneous depending on several factors including the consumer's metabolism, concentration of the synergistic components consumed, and the activity before, during and after the consumer has consumed these beverage formulations.

In addition, the hordenine stabilizes the β-PEA in that the β-PEA remains in its pre-consumed form for an extended length of time, normally several hours. Without the addition of hordenine the β-PEA would destabilize and become metabolically consumed much more rapidly.

The β-PEA is a stronger NDRI (Norepinephrine-Dopamine Reuptake Inhibitor) and is a weaker-acting SSRI (Selective Serotonin Reuptake Inhibitor) in terms of providing this type of activity across the blood-brain barrier when consumed by the consumer.

The synergistic action of β-PEA with hordenine and caffeine provide prolonged mental and physical benefits and diminish or eliminate unwanted withdrawal symptoms after consumption by reduction of adrenaline and dopamine perturbations.

In developing these capsule formulations, it has been determined that there are several method associated with making one or more nutraceutical capsule formulations for increasing thermogenesis. One such method comprises providing at least three distinct synergistic components including caffeine, β-phenylethylamine, and hordenine, such that the components are provided by adding to a container each of the components in selected amounts providing specific concentration(s) and grinding these components in a mortar thereby creating homogenaity of the powdered components forming a basis for the formulations.

Additionally, components including sugar, sugar substitutes, and B vitamins, can be added to capsule and or tablet formulations thereby achieving final formulation compositions for mammalian consumption;

wherein the B vitamins provide additional cognitive enhancing attributes including mental clarity, concentration and stamina;

wherein the three distinct synergistic components are combined with a B vitamin complex, the vitamin B complex including B₃, B₅, B₆, B₇, B₉, and B₁₂mixed together in any combination and in a concentration that will further reduce or eliminate side effects associated with any of the three distinct components and the vitamin B complex with the beverage formulations during or after human consumption;

and, wherein the three distinct synergistic components and one or more B vitamins are further combined with one or more sweeteners including sugars and/or a sugar substitutes.

The composition for the tablet/capsule form of the beverage may or may not include any of the B-vitamins, and may specifically include Vitamin B12.

These final capsule formulation compositions are used to improve functional conditions in mammals associated with mental clarity, concentration, and stamina.

In a further embodiment, the mammals are humans.

Consumption of these nutraceutical beverage formulations includes an administration route selected from the group consisting of oral buccal, sublingual, and combinations thereof.

Most specifically, this disclosure provides for one or more compositions that cause an increase in thermogenesis in mammals, comprising at least caffeine, hordenine and β-phenylethylamine. The caffeine is often sourced from green coffee bean extracts and normally comprises from 0.0001 g to 0.30 g of caffeine from 0.00001 g to 0.1 g of hordenine sourced from barley and from 0.00001 g to 1.0 g β-phenylethylamine sourced from cocoa beans.

### BRIEF DESCRIPTION OF THE DRAWINGS

FIG. 1 is a Venn diagram providing neurotransmitter relationships of serotonin, norepinephrine, and dopamine as a result of the use of caffeine

FIG. 2 is a Venn diagram providing a description of the known caffeine side effects

FIG. 3 is a plot indicating extracellular concentrations of dopamine (DA) in the shell of the NAc after intraperitoneal administration of saline or caffeine

FIG. 4 is a plot indicating the influence of TAAR1 activation by β-PEA on the uptake function of the monoamine transporters in the cells.

FIG. 5 is a plot indicating that the Trace Amine-Associated Receptor 1, TAAR1, is activated in the presence of the trace amines β-phenylethylamine (β-PEA), tyramine (TYR), tryptamine (TRY), and octopamine (OCT).

FIG. 6 is a schematic illustrating the competitive inhibition of MAO-B by hordenine

### DESCRIPTION

The synergistic capsule formulations providing enhanced thermogenesis together with enhanced mental concentration and stamina and reduced withdrawal (or undesirable physical affects) symptoms contain at least three distinct components. These components include caffeine, hordenine, and β-phenylethylamine. Each of these components have been carefully chosen so that their enhanced synergistic effects can be combined and regulated to ensure a decrease or elimination of undesirable side effects associated with each of these “active” substances.

The simultaneous or combined use of β-phenylethylamine (endogenous in the brain—crosses the blood-brain barrier) and hordenine, where hordenine is provided as an MAO-B inhibitor that prevents the rapid peripheral metabolism-breakdown of β PEA. However, since hordenine is a MAO-B inhibitor it can also prevent the metabolism of dopamine, norepinephrine, and epinephrine as well. The result provides for relative stability of the norepinephrine levels that reach the brain, thereby reducing the possibility of a rapid change resulting in undesirable withdrawal symptoms. Adding caffeine to assist with both uptake and release of neurotransmitters, the present disclosure describes a capsule formulation that enhances many aspects of mental concentration and stamina.

In order to fully comprehend the ingenuity associated with these capsule formulations, each of the three distinct components and how they interact with each other to enhance physical and mental aspects of the human condition by also lessening or eliminating undesirable side effects, are described in careful detail below.

Caffeine

For the capsules described in this disclosure, the caffeine is derived primarily from green coffee bean extract. It is also possible to provide synthetic caffeine. As with all psychoactive drugs, caffeine achieves its effects by imitating or altering the release or uptake of neurotransmitters, the chemical messengers that direct how the neurons of the CNS (central nervous system) interact with each other. Neurotransmitters are altered by drugs in a variety of ways, including increasing or decreasing their synthesis, inhibiting or enhancing their transport, modifying their storage, release, or the way they are degraded, or simply by directly mimicking their activity or, alternatively, by blocking their action at the receptor site.

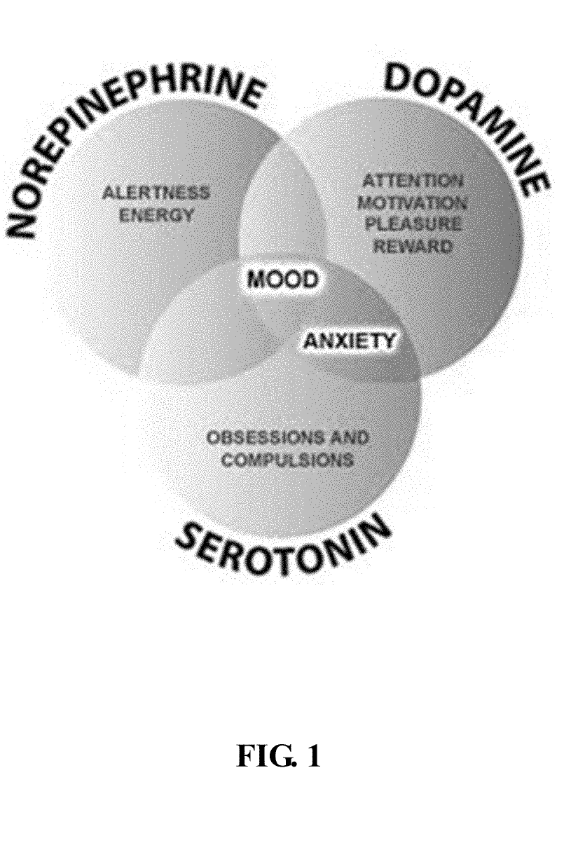

Caffeine achieves many of its effects by blocking the activity of adenosine, a neurotransmitter that affects almost every bodily system. Because one of the primary actions of adenosine is to activate drowsiness and sleep, caffeine, by blocking the uptake of adenosine, keeps us from feeling the effects of fatigue. As the Venn Diagram (FIG. 1) indicates, scientists have learned that, largely as a consequence of its blockade of adenosine receptors, caffeine also has profound effects on most of the other major neurotransmitters, including dopamine, acetylcholine, serotonin, and, in high doses, norepinephrine. By affecting these other neurotransmitters, caffeine is able to deliver energy in the form of thermogenesis to boost capacities even when humans are well-rested, something that could not be explained by the inhibition of adenosine alone. By increasing the transmission of dopamine, caffeine improves mood and there is evidence that it protects brain cells from age and disease related degeneration. By increasing the activity of acetylcholine, caffeine increases muscular activity. By serotonin levels, caffeine relieves depression and provides brain stimulation leading to relaxation, alertness and has even shown relief from migraine headaches. Though caffeine may lower serotonin levels, the synergistic effects of the formulation of this disclosure including β-phenylethylamine and hordenine mitigate or eliminate this caffeine-induced reduction of serotonin.

Studies have shown that caffeine binds to and inhibits adenosine receptors in the brain. Normally, when adenosine binds to these receptors, drowsiness occurs. Caffeine effectively prevents this response and instead allows for alertness. In addition, studies have shown that caffeine induces dopamine release in the brain thereby providing a synergistic effect with β-phenylethylamine. In addition, research suggests that caffeine upregulates dopamine receptors in the brain.

Summarizing, caffeine, by acting to modifying and regulate a host of the body's neurotransmitters, enables humans to achieve enhanced capabilities in four major areas:

Cognitive:

Sharpens reasoning, memory, verbal fluency, concentration, and decision-making and heightens sensuous perception.

Affective:

Enhances moods, increases relaxation, relieves boredom, boosts self-confidence.

Physical:

Improves speed, endurance, strength, and reaction time by increasing thermogenesis, associated with fat burning and metabolic rate.

Therapeutic:

Protects body cells and especially brain cells from some kinds of long-term damage and delivers many other specific therapeutic benefits including pain relief and protection from the pulmonary complications of smoking and the damage from strokes.

Common Side Effects for Caffeine

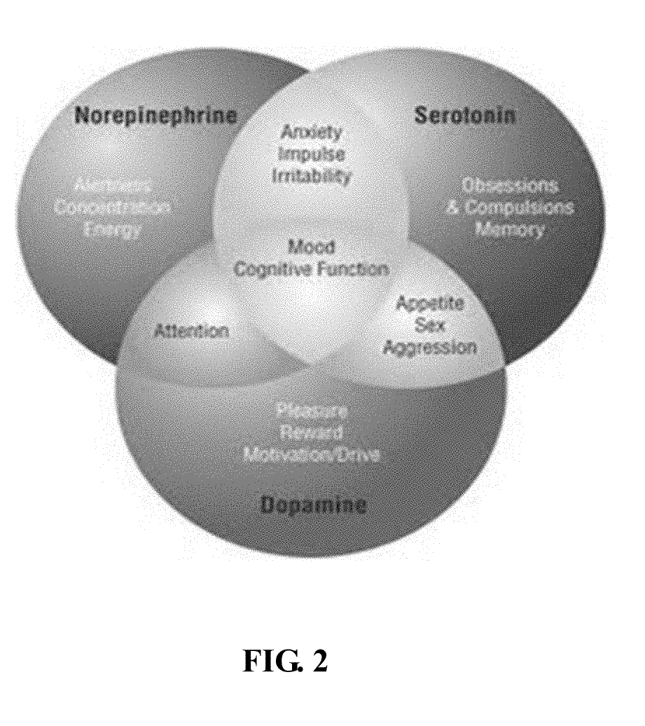

As shown in the Caffeine Side Effects Venn Diagram (FIG. 2), the symptoms associated with withdrawal from caffeine can cause mild to clinically signficant distress or impairment in daily functioning. Mild to increasingly severe physical dependence and withdrawal symptoms may occur upon abstinence, with greater than 100 mg caffeine per day. Some symptoms associated with psychological dependence may also occur during withdrawal. Caffeine dependence also is associated with withdrawal symptoms such as fatigue, headache, irritability, depressed mood, reduced contentedness, inability to concentrate, sleepiness or drowsiness, stomach pain, and joint pain. It has also been reported that withdrawal headaches are experienced by roughly half of those who stop consuming caffeine for two days following an average daily intake of 235 mg.

One of the problems with consuming caffeine in coffee is the sensation of withdrawal. When the body metabolizes caffeine, the initial effects of caffeine will resolve. Now with increased adenosine receptor expression without cafkine inhibition, a person may suffer fatigue and headaches from increased drowsiness and vasodilation induced by adenosine. Caffeine affects people differently. Some people can consume a moderate amount—about two 12-ounce cups of coffee—without experiencing adverse side effects, according to MedlinePlus.

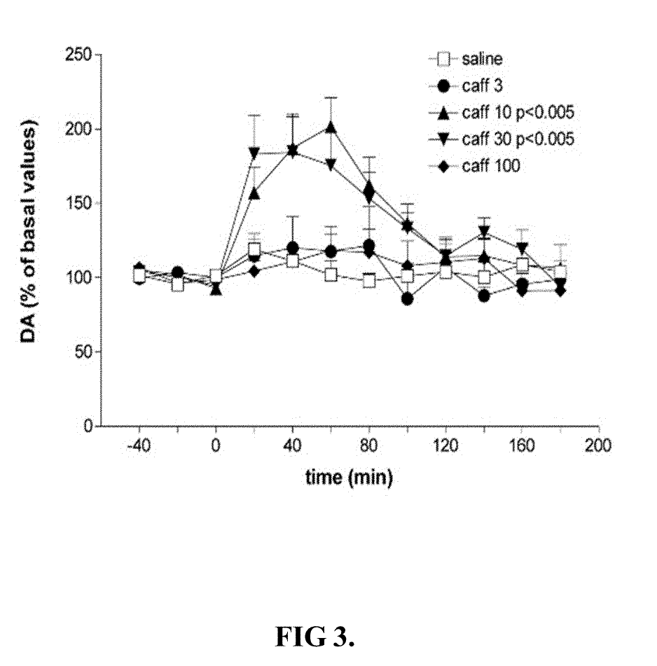

Studies have shown that caffeine binds to and inhibits adenosine receptors in the brain. Normally, when adenosine binds these receptors, we feel drowsiness. Caffeine effectively prevents this response and, instead, we feel wakeful. Aside from caffeine's effect with adenosine receptors, studies have shown that caffeine may induce dopamine release in the brain, thus having a synergistic effect with β-phenylethylamine. Additionally, it appears that caffeine upregulates dopamine receptors in the brain. FIG. 3 provides data to this effect.

β-Phenylethylamine

Phenethylamine (PEA), also known as β-phenylethylamine (β-PEA) or 2-phenylethylamine is an organic compound and a natural monoamine alkaloid, a trace amine, and also the name of a class of chemicals with many members that are well known for their psychoactive and stimulant effects.

The known chemical structure (1) is shown below;

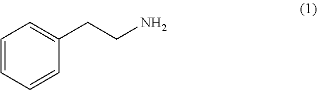

β-phenylethylamine functions as a monoaminergic neuromodulator or “mesencephalic enhancer” and, to a lesser extent, a neurotransmitter in the human central nervous system. It plays a key role in the functioning of our innate and acquired drives. There are enhancer-sensitive neurons in the brain that work in a split second on a high activity level due to the use of β-PEA. In mere microseconds, β-PEA causes an impulse-mediated release of catecholamines (dopamine and epinephrine) and serotonin in the brain. This causes rapidly occurring improvements in cognitive performance, attention, awareness, pleasure, libido, and it provides a sense of wellbeing. According to the pioneering research of Dr. Joseph Knoll, a respected neurochemist, pharmacologist, and emeritus professor, catecholamine levels (neurotransmitters such as dopamine and norepinephrine) reach maximum levels at sexual maturity in humans and then begin a long, gradual reduction. The rate of decline at least in part determines how rapidly a person ages. According to Dr. Knoll, catecholamine levels, learning ability, sexual activity, and longevity are all interlinked. It has been postulated that the efficiency of catecholarnine brain machinery plays a major role in determining quality and duration of human life. Higher-performing, longer-living individuals have a more active, more slowly deteriorating catecholamine system than their lower-performing, shorter-living peers. The boost of energy-generating catecholamines (dopamine and norepinephrine) produces amphetamine-like stimulation and performance enhancement. β-PEA is potent in promoting the efflux of catecholamines and blocking their re-uptake, especially that of dopamine. β-PEA also decreases the reuptake of serotonin albeit less efficiently. That means that β-PEA is a strong NDRI (Norepinephrine-Dopamine Reuptake Inhibitor) and a weaker-acting SSRI (Selective Serotonin Reuptake Inhibitor). Their synergistic actions make β-PEA an ideal nutraceutical complement or alternative to standard protocols and is one purpose of incorporating the use of hordenine, as further described below.

It has also been recognized that β-PSA's ability to elevate blood catecholamine levels may be useful in the thermogenic burning of stored body fat for losing weight. Increased levels of epinephrine and norepinephrine can stimulate beta-adrenergic receptors located on adipose (fat) tissue to release fatty acids into circulation as a fuel source. In other words, β-PEA, as with caffeine, can be used to increase the metabolism and further burn fat for energy. In addition, catecholamines act on hormone-sensitive lipase, the enzyme for removing fat from storage sites.

Starting around age twenty-five, there is a lifelong decline in catecholamine neurotransmitters (epinephrine, norepinephrine, and dopamine), a slower decline in the indoleamine neurotransmitter (serotonin), and a shifting imbalance of the catecholamine/serotonin ratio. Catecholamine deficiencies and neurotransmitter imbalances are a principal cause of loss of “hypothalamic sensitivity” for the progressive metabolic shifts that produce aging and the diseases of aging, according to Dr. Vladimir Dilman's Neuroendocrine Theory of Aging. β-PEA induces behavioral and physiological effects similar to those of amphetamine. Unlike amphetamine and other stimulants, researchers have referred to β-PEA as an “endogenous amphetamine” because the brain produces it. After ingesting β-PEA humans commonly report a surge of energy, wakefulness, alertness, attention and heightened senses.

According to Dr. Dilman, a renowned Russian bio gerontologist, aging is caused by a progressive loss of sensitivity by the hypothalamus (and related structures in the brain) to feedback inhibition from hormones and neurotransmitters. Throughout one's lifespan, this loss of sensitivity produces a progressive shilling away from internal balance and altered levels of hormones, neurotransmitters, and cell signalers. These are the cause of many post-maturational diseases, accelerated aging, and earlier death. The Neuroendocrine Theory of Aging explains in detail how this causes the major diseases of aging, which contribute to over 85 percent of early deaths of middle-aged and elderly individuals.

To correct catecholamine deficiencies to help delay aging, prolong life span, prevent aging disorders, and restore youthful biological functions, Dr. Dilman and other researchers in the field of anti-aging have suggested the following:
1. Increase neurotransmitter production and activity;
2. Decrease catecholamine breakdown from MAO-B enzymes;
3. Correct neurotransmitter deficiency and imbalance of the catecholamine/serotonin ratio;
4. Inhibit neurotransmitter re-uptake, to increase inter-synaptic neurotransmitter levels
5. Correct the decrease in receptor sensitivity and responsiveness of target cells and tissues to neurotransmissions.

Current research on β-PEA trace amine receptor neuro-modulator and neurotransmitter actions is reaping clinical rewards. β-PEA is proving beneficial for attenuating attention-related problems, controlling addictions, overcoming substance abuse, and correcting neurobehavioral problems. The success rate for treating depression with β-PEA has been shown to be equivalent to the effective percentage for the major Serotonin-Selective Re-uptake inhibitors (SSRI's)—but without their serious side effects and toxicity. In fact, β-PEA has produced sustained relief of both acute and chronic depression in a significant number of people, including some who were unresponsive to standard protocols, according to research psycho-pharmacologist and psychiatrist Dr. Hector Sabelli. These are ongoing and exciting areas of research for β-PEA with many practical uses and clinical implications.

β-PEA is biosynthesized from the amino acid L-phenylalanine by enzymatic decarboxylation via the enzyme aromatic L-amino acid decarboxylase. In addition to its presence in mammals, β-phenethylamine is found in many other organisms and foods, such as chocolate, cauliflower, kale cabbage and acacia, especially after microbial fermentation. It is sold as a dietary supplement for purported mood and weight loss-related therapeutic benefits; however, orally ingested phenethylamine experiences extensive first-pass metabolism by monoamine oxidase B (MAO-B) and then aldehyde dehydrogenase (ALDH), which metabolize it into phenylacetic acid. This prevents significant concentrations from reaching the brain when taken in low doses.

As an endogenous stimulant of the human (and other mammals) brain, β-PEA amplifies the activity of major neurotransmitters thereby providing increased longevity, slower aging, higher performance, a sense of well-being, and a renewed youthful-functioning body. β-PEA increases the actions of dopamine (for well-being and feeling pleasure), norepinephrine (the brain's stimulant for wakefulness and higher performance), acetylcholine (for improving memory and mental activity), and serotonin (for better mood emotion and impulse control). β-PEA is a highly-concentrated neurotransmitter in the limbic system (the brain's emotional center) that increases motivation, physical drive, feelings, and social activity.

β-PEA is the parent compound of I-deprenyl, a catecholamine-enhancing, dopamine-increasing, and neuro-protective compound with proven life extension actions in animal research. L-deprenyl produces a concentration spike in brain β-PEA. β-PEA releases acetylcholine, a neurotransmitter that plays an integral role in learning and memory. Brain receptors respond to acetylcholine by facilitating memory and higher cognitive functions. In addition, β-PEA increases noradrenaline action and causes release of the catecholamines which includes dopamine and epinephrine (adrenaline) required for alertness and concentration. It has also been shown that an increase in glutamate from β-PEA is involved with brain circuitry that helps form the neural networks associated with memories.

How β-PEA Functions in the Brain and Body

β-PEA readily crosses the blood-brain barrier. It is rapidly available in the brain to increase neurotransmission by blocking neurotransmitter (catecholamines and indoleamine) reuptake, as a regulator of neurotransmitter transport and efflux, and an excitatory (stimulating) neurotransmitter in different region of the brain.

Amplifies Neurotransmitter Activity

β-PEA is released from nerve vesicles in the brain, causing an efflux of neurotransmitters in response to a given nerve signal, therefore providing amplification of nerve cell activity. β-PEA induces higher concentration, continuous robust efflux, and greater availability of dopamine (for feeling pleasure and wellbeing), norepinephrine (the brain's stimulant creating energy for movement), acetylcholine (for memory and cognitive functions), and serotonin (mood enhancement, and impulse control).

Modulates Neurotransmitter Functions by Binding TAAR1

β-PEA modulates neuro-transporter functions by binding with its paired Trace Amine-Associated Receptor 1 (TAAR1). TAAR1 is a G-protein coupled receptor that is activated by β-PEA and certain monoamines. Activation of TAAR1 by β-PEA significantly inhibits the uptake and induces efflux of the neurotransmitters dopamine, norepinephrine, and serotonin. β-PEA increases the extracellular levels of these neurotransmitters by inhibiting their reuptake into the pre-synaptic cell, and this increases their ability to provide beneficial activity throughout the body. Trace amines, including β-phenylethylamine (β-PEA), tyramine, tryptamine, and octopamine, have been known to be heterogeneously distributed in mammalian brain tissues, and their distribution spatially parallels the origins and terminal projection areas of the monoaminergic neurons. In monoaminergic neurotransmission, common biogenic amines, including dopamine, norepinephrine and serotonin, are well established as neurotransmitters, which are synthesized from their precursor amino acids in neurons, stored in vesicles of neuronal terminals and released into the synaptic clefts to interact with both presynaptic and postsynaptic receptors. Although trace amines share similarities with common biogenic amines in structure, metabolism, and distribution, their roles and functional profiles in the brain are far from being as clear as those for common biogenic amines.

With high turnover rates, trace amines are dynamically regulated by enzymes and are present in the brain at generally low concentrations. No trace amine specific neurons have been discovered and were once thought of as metabolites or faux neurotransmitters. However, the rate in synthesis of trace amines is equivalent to that of dopamine and norepinephrine, and altered levels of the trace amines are associated with various neuropsychiatric disorders, which are hallmarked by changes in monoaminergic activity. These properties and amphetamine-like effects of the trace amines suggest that trace amines may serve as neuromodulators in the brain.

It has been confirmed that TAAR1 is widely expressed in the rhesus monkey brain (mammalian model), notably in monoaminergic nuclei and that rhesus monkey TAAR1 responds to a wide spectrum of chemicals, including trace amines. However, the functional interaction of TAAR1 with trace amines and the role that TAAR1 plays in the brain remain unclear. Previous data showed that TAAR1 signaling is enhanced by monoamine transporters and that TAAR1 activation can modulate DAT kinetics in vitro. Data collected by several investigators also showed co-localization of TAAR1 and DAT in a subset of dopamine neurons in the rhesus monkey and mouse substantia nigra. It was also observed that TAAR1 expression in norepinephrine transporter (NET)-positive neurons in the locus coeruleus of the rhesus monkey

A 2008 study by Lindemann et al. (2008) reported TAAR1 expression in the dorsal raphe nucleus. These findings suggest that TAAR1 may serve as a presynaptic regulator in functional modulation of monoamine transporters. With respect to monoamine autoreceptors, it has been reported that their activation by monoamine neurotransmitters provides feedback regulation of monoamine neurotransmitter release but it is unknown whether trace amines exert any effects via monoamine autoreceptors. In a more recent study, the interaction of trace amines with TAAR1 and monoamine autoreceptors were investigated together with whether monoamine autoreceptors influence TAAR1 signaling and monoamine transporter function in response to trace amines.

This study used transfected HEK293 cells and brain synaptosomes derived from nonhuman primates, wild-type mice, and TAAR1 knockout mice to investigate the effects of β-PEA activation of TAAR1 on the uptake and efflux function of DAT, NET, and serotonin transporter (SERT). The data confirm that all the trace amines tested recognize and excite TAAR1 and reveal that trace amines do not interact with monoamine autoreceptors to alter TAAR1 activity. The data also revealed that β-PEA alters monoamine transporter function via interacting with TAAR1 but not monoamine autoreceptors, which may suggest a common mechanism by which trace amines exert a modulatory role on monoamine transporters in the brain.

Homeostatic Regulator Guarding Against Metabolic Dysfunctions

β-PEA is also a neural guardian of homeostasis (maintaining healthy metabolic equilibrium). The binding of β-PEA with TAAR1 protects delicate neural circuitry against harmful changes. β-PEA self-regulates transmitter activity to prevent over-excitation or under-stimulation transmitter signal strength and activity. β-PEA acts as a homeostatic controller to maintain the neuronal activity within defined physiological limits to prevent metabolic dysfunctions and neurological disorders. This makes β-PEA and other trace amines perfect candidates for the development of novel therapeutics for a wide range of human disorders.

Endogenous Amphetamine-Like Stimulating Neurotransmitters

β-PEA is an excitatory neurotransmitter with its own receptor and a chemical structure similar to amphetamines that induces behavioral and electrophysiological effects similar to those of amphetamine. Ever, as mentioned above, unlike amphetamine, β-PEA is endogenous to the brain and does not develop tolerance or dependency, or produce any side effects, such as amphetamines sold under the trade name Adderall® and the adverse effects of the popular drug stimulant Ritalin® that is prescribed for treating Attention Deficit Disorders.

β-PEA may trigger neurotransmitters for “brain plasticity” and “neurogenesis” (the forming of new brain cells, information processing connections, and functions) that increase cognition, learning, memory, skills, smartness, and performance. The mechanism is thought to involve β-PEA's action of increasing dopamine neurotransmitters from synapses and acting as a dopamine re-uptake inhibitor in certain brain regions.

In summary, β-PEA has been found to act as a neurotransmitter by;
Preventing the reuptake and promoting efflux of dopamine which enhances pleasure, libido and emotional wellbeing.
Increasing epinephrine and norepinephrine catecholamine at nerve terminals, for energy production and inhibition of their re-uptake.
Increasing the action of acetylcholine (Decreasing the reuptake of acetylcholine) for cognitive functions by stimulating the AMPA glutamatergic receptors.
a Elevating mental alertness and mood by suppressing the inhibitory effects of GABA-B receptors.
Preventing the reuptake and promoting efflux of serotonin which causes uplifting activity on mood, emotions, and control.

Furthermore, β-PEA regulates neurotransmitter activity to prevent aberrant neurosignaling. Thus β-PEA acts as a homeostatic regulator to maintain the neuronal activity of monoamine neurotransmitters within defined physiological limits.

Convincing evidence has been presented for using β-PEA in the treatment for a wide range of neurological dysfunctions and behavioral disorders, such as:
Affective disorders (depression)
Attention deficit/hyperactivity disorder
Cognitive dysfunction (brain fog, confusion, forgetfulness, poor concentration, a sluggish cognitive tempo, slowed reaction time, and diminished awareness)
Drug abuse and substance dependence (alcoholism, nicotine dependence, and addictions to methamphetamines, cocaine opioids, and psycho-stimulants)
Addictive behavior (gambling, sexual addiction)
Eating disorders (obesity, anorexia)

The human brain forms β-PEA from the essential amino acid 1-phenylalanine by an enzyme-driven cellular process. Phenylalanine is the precursor to the amino acid tyrosine, which produces the neurotransmitters dopamine, norepinephrine, and adrenaline in a sequential process, but phenylalanine supplements don't significantly boost β-PEA concentrations. It has been found that phenylalanine supplements can boost catecholamine neurotransmitter levels excessively, producing undesirable side effects including anxiety, headaches, and hypertension. In some cases phenylalanine has been shown to transform into neurotoxic (brain-damaging) metabolites, By contrast, β-PEA safely increases and amplifies the activity of dopamine, norepinephrine, and other brain transmitters to produce desirable and remarkable effects.

As also mentioned above, the human body can synthesize significant quantities of β-PEA, however, functional concentrations of β-PEA remain low due to its rapid metabolism via MAO-B (monoamine oxidase B). As result, β-PEA effects are not sustained. The beverage formulations of the present disclosure have been designed through a synergistic combination to increase the half-life of β-PEA allowing for sustainable effects.

Common Side Effects for β-PEA

Unlike drug stimulants that are highly addictive and harmful to your health, β-PEA produces non-toxic and non-addictive stimulation with limited side effects. β-PEA protects neurons and does not overstimulate the nervous system. β-PEA doesn't deplete neurotransmitter levels, it modulates them. This avoids the “crashing upon cessation of use” that is common with stimulant drugs. In terms of safety, β-PEA does produce the adverse effects of pharmaceutical NDRI's and SSRI's. This is due to β-PEA modulation of neurotransmissions and its intrinsic neuro-protective properties.

β-PEA does not involve habituation and produces limited side effects. β-PEA has been referred to by scientists as an “endogenous amphetamine” because it is produced naturally by the brain. Following ingestion of β-PEA people commonly report a surge of energy, wakefulness, alertness, and heightened senses. Because β-PEA is endogenous to the brain it seems to be a safer alternative to amphetamines sold under the trade name Adderall® and the adverse effects of the popular drug stimulant Ritalin® that are prescribed for treating Attention Deficit Disorders.

A single side effect which could be deleterious to health is that β-PEA has demonstrated appetite-reducing activity, reducing food intake in animal research.

In summary, β-phenylethylamine (β-PEA) is a trace monoamine produced endogenously (naturally) in the brain. It is synthesized from the modification of phenylalanine (amino acid) just like the neurotransmitters dopamine epinephrine, and norepinephrine (serotonin is synthesized from tryptophan). Current research shows that PEA decreases the reuptake of dopamine, serotonin, and norepinephrine causing enhancement of mood, energy and focus.

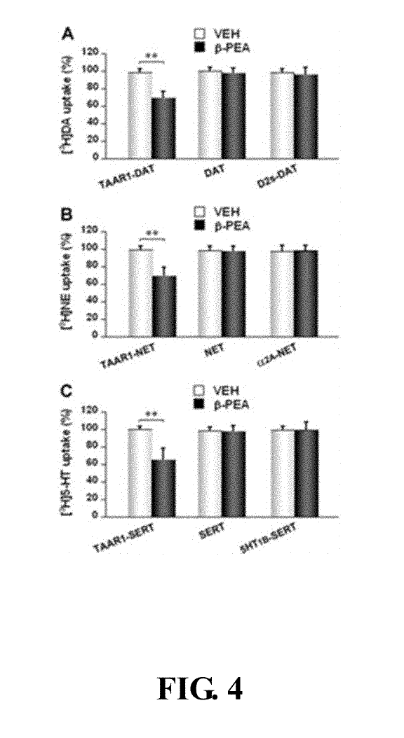

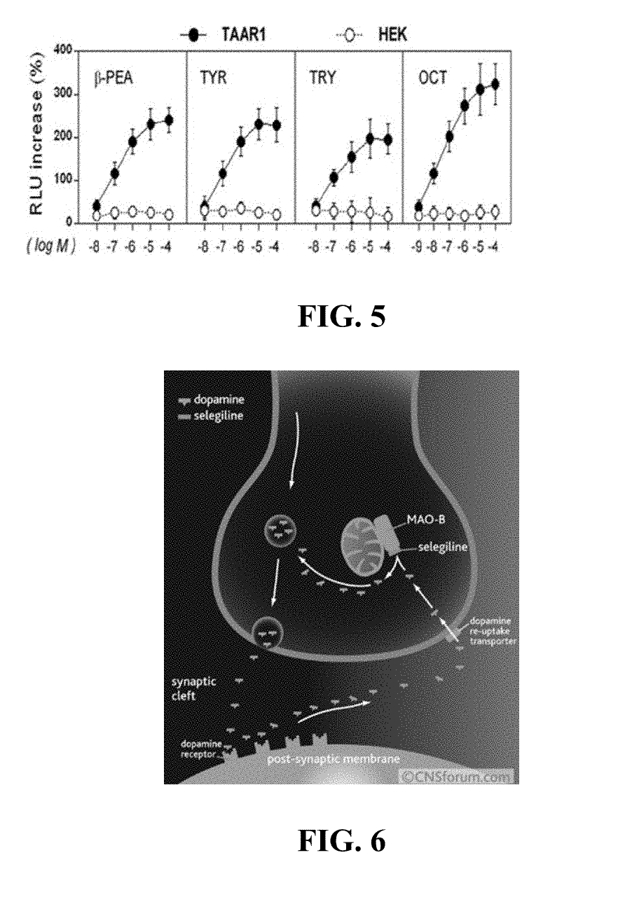

In the presence of β-PEA, there is less dopamine reuptake (more dopamine at nerve synaptic cleft). The data in FIGS. 4 and 5 indicate this effect for Rhesus monkeys.

FIG. 4 shows that βphenylethylamine (β-PEA) does not directly interact with the monoamine transporters DAT (dopamine transporter), NET (norepinephrine transporter), or SERT (serotonin transporter). β-PEA does, however, does activate TAAR1, which subsequently modulates monoamine transporter function downstream. To demonstrate this, three cell lines were generated for each monoamine. For dopamine: a cell line expressing TAAR1 and DAT, a cell line expressing only DAT, and cell line expressing only D2s-DAT (a subtype of DAT) with the latter two being controls is shown. This was repeated for norepinephrine, serotonin and the respective transporters as well. Transporter activity with and without treatment with β-PEA was measured by determining the amount of tritium-labeled monoamine present inside the cells after treatment with stock solutions of the respective monoamines. The data indicates that β-PEA reduces the uptake of the monoamines by their respective transporters.

FIG. 5 shows that the Trace Amine-Associated Receptor 1, TAAR1, is activated in the presence of the trace amines β-phenylethylamine (β-PEA), tyramine (TYR), tryptamine (TRY), and octopamine (OCT). This activation results in an increase of cyclic adenosine monophosphate (cAMP) via activation of adenylyl cyclase. In order to determine this effect of the trace amines TAAR1, HEK293 cells were transiently transfected with a rhesus monkey TAAR1 expression construct as well as CRE-Luc and pGL4.73 reporter systems to determine cAMP production. The data confirms β-PEA, TYR, TRY, and OCT activates TAAR1 whereas the control HEFT cells did not produce cAMP.

Hordenine

Hordenine N,N-dimethyltyramine is an alkaloid of the phenethylamine class that occurs naturally in a variety of plants, taking its name from one of the most common, barley (Hordeum species). Chemically, hordenine is the N-methyl derivative of N-methyltyramine, and the N,N-dimethyl derivative of the well-known biogenic amine tyramine from which it is biosynthetically derived and with which it shares some pharmacological properties that are described below.

Hordenine is known as a stimulant of the central nervous system that causes a release of norepinephrine, and has the ability to promote weight loss by enhancing metabolism.

The known chemical structure (2) is shown below;

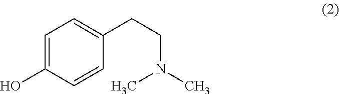

Hordenine is also a highly selective substrate of MAO-B and acts as a temporary reversible MAO-B inhibitor. Because hordenine crosses the blood-brain barrier it is able to inhibit MAO-B enzymes in both the body and brain. The method of action for β-PEA s different in that the human body can synthesize significant quantities of β-PEA, however, functional levels of β-PEA remain fairly low because it is usually broken down by the enzyme MAO-B within several hours. Hordenine is also antiasthmatic, antidiarrheal, antifeedant, antispasmodic, bronchorelaxant, cardiotonic, hepatoprotective, sympathicomimetic, and acts as a vasoconstrictor.

In comparison, caffeine can act as either a vasoconstrictor or vasodilator depending on dose and which areas of the body are being acted upon. This fact assists in providing synergies between β-PEA, hordenine and caffeine (another synergistic effect between caffeine and β-PEA is that caffeine seems to reduce serotonin levels whereas β-PEA decreases the reuptake-increasing serotonin at the nerve terminal).

The first report of the isolation from a natural source of the compound hordenine was made by Arthur Heller in 1894, who extracted this alkaloid from the cactus Anhalonium fissuratus (now reclassified as Ariocarpus fissuratus), naming it “anhalin”. Twelve years later, E. Léger independently isolated an alkaloid which he named hordenine from germinated barley (Hordeum vulgare) seeds. Ernst Späth subsequently showed that these alkaloids were identical and proposed the correct molecular structure for this substance, for which the name “hordenine” was ultimately retained.

Hordenine is present in a fairly wide range of plants, notably amongst the cacti, but has also been detected in some algae and fungi. It occurs in grasses, and is found at significantly high concentrations in the seedlings of cereals such as barley (Hordeum vulgare) (˜0.2%, or 2000 μg/g), proso millet (Panicum miliaceum) (˜0.2%), and sorghum (Sorghum vulgare) (˜0.1%). Reti, in his 1953 review of naturally-occurring phenethylamines, notes that the richest source of hordenine is the cactus Trichocereus candicans (now reclassified as Echinapsis candicans), which was found to contain 0.5-5% of the alkaloid.

It is known that hordenine is biosynthesized by the step-wise N-methylation of tyramine, which is first converted to N-methyltyramine, and which, in turn is methylated to hordenine. The first step in this sequence is accomplished by the enzyme tyramine N-methyltransferase (tyramine methylpherase), but it is uncertain if the same enzyme is responsible for the second methylation that actually produces hordenine,

It should be noted that the “methyl hordenine HCl” which is listed as an ingredient on the labels of some nutritional supplements is in all likelihood simply hordenine hydrochloride, since the description of “methyl hordenine HCl” given by virtually all bulk suppliers of this substance corresponds to that for hordenine hydrochloride (or possibly just hordenine). There are five regioisomeric compounds that would correspond to the name “methyl hordenine HCl”, if it were interpreted according to the rules of chemical nomenclature: α-methyl hordenine, β-methyl hordenine, 2-methyl hordenine, 3-methyl hordenine, and 4-O-methyl hordenine—each in the form of its HCl salt.

In a 1995 study, Hapke and Strathmann reported that in dogs and rats hordenine produced a positive inotropic effect on the heart (i.e. increased the strength of contraction), increased systolic and diastolic blood pressure, and increased the volume of peripheral blood flow. Movements of the gut were inhibited. Additional experiments on isolated tissue lead these investigators to conclude that hordenine was an indirectly acting adrenergic agent that produced its pharmacological effects by releasing stored norepinephrine (NE).

In a study of the effects of a large number of compounds on a rat trace amine receptor (rTAR1) expressed in HEK 293 cells, it was found that hordenine, at a concentration of 1 μM, had almost identical potency to that of the same concentration of β-phenethylamine in stimulating 3′,5′-cyclic adenosine monophosphate (CAMP or cyclic AMP) production through the rTAR1. cAMP is a “second messenger” important for many biological processes including intracellular signal transduction in many organisms. The potency of tyramine in this receptor preparation was slightly higher than that of hordenine.

cAMP is a second messenger important in many biological processes cAMP is a derivative of adenosine triphosphate (ATP) and used for intracellular signal transduction in many different organisms, conveying the cAMP-dependent pathway,

Common Side Effects for Hordenine

In experimental animals, given sufficiently large doses parenterally (i.e. by injection) of hordenine produced an increase in blood pressure, as well as other disturbances of the cardio-vascular, respiratory, and nervous systems.

Modern studies were carried out by Frank and co-workers, who reported that intravenous (IV) administration of 2 mg/kg of hordenine to horses produced substantial respiratory distress, increased the rate of respiration by 250%, doubled the heart rate, and caused sweating without changes in basal body temperature or behavior. All effects disappeared within 30 minutes. The same dose of hordenine given orally did not produce any of the effects seen after parenteral administration.

Combining Hordenine with other MAO inhibitors or pharmaceutical drugs for depression and anxiety is ill advised. Most of the following side effects only occur at much higher than recommended doses and include: nausea, insomnia, dizziness, anxiety, upset stomach, mood swings, rapid heart rate, and possibly hallucinations.

In summary, hordenine is a substrate of monoamine oxidase B (MAO-B). When taken with β-phenylethylamine (PEA), hordenine effectively prevents MAO-B from rapidly metabolizing β-PEA and dopamine. MAO-B inhibition increases the half-life of β-PEA and dopamine resulting in prolonged effect with no sudden crash,

FIG. 6 illustrates how the inhibition of MAO-B by hordenine prevents the rapid metabolism (increases half-life) of dopamine thereby subsequently allowing for accumulation of dopamine at the synaptic cleft that controls neuron transport to the brain as well as to other organs.

The nutraceutical capsule formulations of the present disclosure combines the scientific understanding of the synergistic effects described in detail above by including at least the three basic components of caffeine, phenylethylamine (β-PEA), and hordenine. These thermogenesis enhancing components provide a consumable product that reduces and in some cases eliminates the sudden “crash” (aka withdrawal symptoms described above) often experienced with leading energy drink brands. The synergistic effect of the ingredients delivers the burst of energy (thermogenesis), increased mental concentration and stamina needed to accomplish daily goals, tasks, and elevate overall human performance.

The composition selected has been carefully with the proper concentrations to provide the optimal dose for maximum effect with minimal side effects to ensure slowing of metabolic uptake and reduction to ensure that there will be little or no residual drowsiness or de-energization.

One such composition is for a “focus blend” capsule formulation wherein this capsule would comprise the following components;

a). 300 mg β-phenylethylamine

c). 50 mg caffeine (anhydrous)

d). 35 mg hordenine HCl

Representative pharmaceutically acceptable calcium salts for tablet inclusion include calcium chloride, calcium tartrate, calcium maleate, calcium lactate, calcium citrate, calcium phosphate, calcium acetate, calcium carbonate, calcium hydrogen carbonate, calcium lactate calcium fumarate, calcium sulfate, calcium bromide, calcium mesylate, calcium palmoate, calcium iodide, calcium nitrate, calcium gluconate and calcium methylsulfate. Alternatively, sodium and magnesium salts of these types may be equally acceptable. Such salts can be purchased for example from Cargill and Esco listed at both http://www.cargill.com/salt/products/industrial/pharmaceutical/cargill-usp-grade/index.jsp and https://www.es co-salt.com/pharma_salt.html?&L=1, respectively.

With respect to ultimate dosing preferences, concentration levels within these capsule formulations are developed based on typical human subjects (e.g. a 70 kg subject). If the present composition is used in other mammals or in various human subjects, it may be necessary to modify the dosage. Modification of dosages based on the needs of the subject is well within the skill of the ordinary artisan. It is therefore understood that these dosage ranges are by way of example only, and that daily administration can be adjusted depending on various factors. The specific dosage of the compounds to be administered, and the duration of treatment are interdependent. The dosage and treatment regimen will also depend upon such factors as the specific components used, the efficacy of the components and the specific attributes required for each of the possible combinations of capsules that can be produced. Compliance with government regulations including packaging and labeling issues provide additional requirements that must be considered in order to properly commercialize these capsules.

A standard operating procedure (SOP) for making capsules of the present disclosure is included below;
1.1.1. The purpose of this procedure is to outline the process for preparing a small batch of Focus Capsules known as capsule formulations in this specification.

2. Scope
2.1.1. This procedure applies to all personnel trained to perform research and develop these formulations.

Procedure

| Responsible Party | Action Step |
|---|---|
| | Preparation |
| Analyst | 1. Assemble Capsule Filling System with size |
| | 00 capsule change parts. |
| | 2. Pour required amount of size 00 capsules |
| | into Orienter and place into Filling System. |
| | 3. Separate the capsules. |
| | Measuring and Mixing Intermediates |
| | 4. Measure out required amount of |
| | intermediates Phenylethylamine (PEA), |
| | Hordenine HCl, and Caffeine Anhydrous into |
| | separate weighing boats and using clean |
| | spatulas or scoops as per the Focus |
| | Capsule formulation. |
| | 5. Pour PEA into mortar and grind crystals into |
| | a fine powder with the pestle. |
| | 6. Pour Hordenine HCl into mortar with PEA |
| | and grind/mix thoroughly. |
| | 7. Pour Caffeine Anhydrous into mortar with |
| | the other intermediates and grind/mix |
| | thoroughly. |
| | Filing the Capsules with the Focus Powder |
| | 8. Place Powder Tray onto Filling System and |
| | pour the Focus powder into the Powder |
| | Tray. |
| | 9. Using the Powder Spreader, spread the |
| | powder to fill all capsules. If some capsules |
| | are not filled, remove them before the next |
| | step. |
| | 10. Remove the Powder Tray and replace the |
| | Locking Plate. |
| | 11. Squeeze the Filling System to raise the |
| | bottom plate and put capsules back |
| | together. |
| | 12. Remove the Locking Plate and Caps Tray |
| | with capsules still inside and gently press |
| | with the Capsule Locker to ensure all |
| | capsules are tightly sealed. |
| | 13. Remove the Locking Plate from the Caps |
| | Tray and empty the Focus capsules into a |
| | labelled bottle. |

In addition to the SOP provided above, there are many additional methods of making specific capsule and tablet formulations. Example 1 below indicates a possible composition for making these capsules. Therefore capsule formulations herein can also be contained and provided in appropriate alternative encapsulating forms.

For the purposes of the embodiments contained herein, a tablet is a mixture of active and inactive ingredients that are generally compressed or “punched” into a desired shape and size. Tablet formulation normally requires powder compression equipment that will allow for shaping the tablet according to the shape of the cavity. Powder fills the cavity and either heat or pressure or both heat and pressure are used to form the tablet and table shape.

Alternatively, a capsule is provided in two shell portions—male and female portions—and are filled and fitted into one another. The shell parts can be made from gelatin, vegetarian and vegan ingredients, and/or other suitable materials for human or mammalian consumption. The capsule can be provided in an immediate-release or extended-release form.

Example embodiments are directed to therapeutic compositions or formulations that include natural ingredients for the enhancement of mental clarity, concentration and stamina in mammals. In particular, according to non-limiting example embodiments, compositions provided herein may include one or more core ingredients, and optionally one or more synergistic ingredients.

### EXAMPLE 1

An example of the components of such a capsule formulation includes the following ingredients to provide a “Focus Capsule” according to Table 1 below,

TABLE 1
Capsule Formulation

| Energy (Shot) | Amount (milligrams) |
|---|---|
| Phenylethylamine (PEA) | 300 |
| Hordenine HCL | 35 |
| Caffeine Anhydrous | 50 |
| Total Weight | 385 mg |

### EXAMPLE 2

A further example of the components of such a capsule formulation includes the following ingredients to provide a “Focus Capsule” according to Table 2 below.

TABLE 2
Capsule Formulation

| Energy (Shot) | Amount (milligrams) |
|---|---|
| Phenylethylamine (PEA) | 400 |
| Hordenine HCL | 40 |
| Caffeine Anhydrous | 60 |
| Total Weight | 500 mg |

The Supplements Facts Panel for the formulation of Example 2 is provided in Table 3.

TABLE 3
Supplement Facts Panel for Example 2
Directions: Take 1-2 capsules for increased
focus. Do not exceed 2 capsules per day.
Supplement Facts
Serving Size: 1 Capsule
Servings Per Container: 15

| Amount Per Serving | % DV |
|---|---|
| Phenylethylamine HCl | 400 mg † |
| Caffeine Anhydrous | 60 mg † |
| Hordenine HCl | 40 mg † |

† Daily Value Not Established
Other Ingredients: None

While various embodiments and individual features of the present invention have been illustrated and described, various other changes and modifications can be made without departing from the spirit and scope of the invention. As will also be apparent, all combinations of the embodiments and features taught in the foregoing disclosure are possible and can result in preferred executions of the invention.

We claim:

1. One or more nutraceutical encapsulated formulations providing enhanced thermogenesis, mental concentration, and stamina to mammals, comprising at least three distinct synergistic components including caffeine, β-phenylethylamine, and hordenine, and wherein said formulations reduce or eliminate undesirable side effects during or after mammalian consumption of said encapsulated formulations.

2. The formulations of claim 1, wherein said formulations are provided in encapsulated form as capsules.

3. The formulations of claim 1, wherein said formulations are provided in tablet form as tablets.

4. The formulations of claim 1, wherein said mammals are humans.

5. The formulations of claim 1, wherein said hordenine is hordenine HCl.

6. The formulations of claim 1, wherein said β-phenylethylamine is β-phenylethylamine HCl.

7. The formulations of claim 1, wherein said synergistic components act to increase catecholamine and indoleamine concentrations and wherein synergism is accomplished as said catecholamine and indoleamine concentrations in a blood stream cross a blood-brain barrier within a brain of a consumer after consumption of said formulations, thereby reducing or eliminating acute symptoms associated with rapid decline of said catecholamine and indoleamine concentrations in said blood stream.

8. The formulations of claim 8 wherein said synergistic components undergo a metabolic breakdown resulting in a controlled decrease in said catecholamine and indoleamine concentrations in said blood stream such that said concentrations revert to a pre-existing basal level.

9. The formulations of claim 1, wherein said synergistic components provide synergy in that β-phenylethylamine decreases serotonin reuptake, hordenine prevents metabolic breakdown of β-phenylethylamine and maintains serotonin levels, while caffeine decreases serotonin levels.

10. The formulations of claim 1, wherein said synergistic component, β-phenylethylamine diminishes reduction of serotonin levels that occur following prolonged consumption of caffeine.

11. The formulations of claim 1, wherein said three distinct synergistic components are further combined with one or more B vitamins.

12. The formulations of claim 1, wherein said B vitamins provide additional cognitive enhancing attributes including mental clarity, concentration and stamina.

13. The formulations of claim 1, wherein said three distinct synergistic components are combined with a B vitamin complex, said vitamin B complex including B₃, B₅, B₆, B₇, B₉, and B₁₂ mixed together in any combination and in a concentration that will further reduce or eliminate side effects associated with any of said three distinct components and said vitamin B complex within said beverage formulations during or after human consumption.

14. The formulations of claim 1, wherein said three distinct synergistic components and one or more B vitamins and/or salts of B vitamins are further combined with one or more sweeteners including sugars and/or a sugar substitutes.

15. The beverage formulations of claim 1, wherein caffeine is a green coffee bean extract.

16. One or more nutraceutical encapsulated formulations providing enhanced thermogenesis, mental concentration, and stamina to mammals, comprising at least three distinct synergistic components including caffeine, β-phenylethylamine, and hordenine, wherein a boost of energy-generating catecholamines from said caffeine including dopamine and norepinephrine produces amphetamine-like stimulation and performance enhancement and wherein β-PEA further promotes an efflux of said catecholamines that blocks re-uptake of said catecholamines and simultaneously decreases reuptake of serotonin.

17. The formulations of claim 16, wherein said uptake of said catecholamines is predominantly that of dopamine.

18. The formulations of claim 17 wherein hordenine stabilizes said β-PEA by reducing metabolic breakdown of β-PEA during and after consumption.

19. The formulations of claim 18, wherein β-PEA acts as a strong NDRI (Norepinephrine-Dopamine Reuptake Inhibitor) and a much weaker-acting SSRI (Selective Serotonin Reuptake Inhibitor).

20. The formulations of claim 16, wherein synergistic action of β-PEA with hordenine and caffeine provide prolonged mental and physical benefits and diminish or eliminate unwanted withdrawal symptoms after consumption by reduction of adrenaline and dopamine perturbations.

21. The formulations of claim 20, said synergistic action of β-PEA with hordenine and caffeine provide prolonged mental and physical benefits and diminish or eliminate unwanted withdrawal symptoms after consumption by elimination of adrenaline and dopamine perturbations.

22. A method of making one or more nutraceutical formulations for increasing mental focus, concentration and stamina, said method comprising providing at least three distinct synergistic components including caffeine, β-phenylethylamine, and hordenine, said components provided by adding to a capsule each of said components in selected amounts providing selected concentration(s) after grinding and mixing said components into a powder, thereby creating homogeneity of said components, thereby forming a basis for said formulations.

23. The method of claim 22, wherein said components, after grinding and mixing said powder, are placed into a cavity and pressurized and/or heated within said cavity thereby forming one or more tablets through pelletizing said powder.

24. The method of claim 23, wherein to said formulations, additional components including sugar, sugar substitutes, B vitamins, colorants, flavorings, thickening agents, stabilizers, and preservatives are added thereby achieving final formulations for mammalian consumption.

25. The method of claim 23, wherein to said formulation, additional components including fillers, can be added thereby achieving a final tablet form for mammalian consumption.

26. The method of claim 22, wherein providing a dietary supplement in a compressed tablet form, is accomplished comprising:
from 5 percent to 80 percent by weight of a combination of at least two tablet excipients selected from the group consisting of certified organic carbohydrates, at least one of the tablet excipients being a dietary fiber, the combination of said tablet excipients acting as a tablet binder, a tablet disintegrant, or both a tablet binder and a tablet disintegrant.

27. The method of claim 22, wherein said formulations are improving functional conditions in mammals including mental clarity, concentration, and stamina.

28. The method of claim 27, wherein said mammals are humans.

29. The method of claim 28, wherein consumption of said nutraceutical formulations includes an administration route selected from the group consisting of oral buccal, sublingual, and combinations thereof.

30. The method of claim 23, wherein said capsule can be provided in an immediate-release or extended-release form.

31. The method of claim 24, wherein said tablet can be provided in an immediate-release or extended-release form.
